(19) United States
(12) Patent Application Publication
(10) Pub. No.: US 2007/0161898 A1
(43) Pub. Date: Jul. 12, 2007

(54) Raw data reprocessing in ultrasound diagnostic imaging

(75) Inventors: Xiaohui Hao, Redmond, WA (US); Andrzej Milkowski, Issaquah, WA (US); Richard Yung Chiao, Kirkland, WA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/330,001

(22) Filed: Jan. 10, 2006

(51) Int. Cl. A61B 8/00

(52) U.S. Cl. 600/443

(57) ABSTRACT

Raw data reprocessing is provided for ultrasound imaging systems. Clip storing and review functions are more versatile by storing ultrasound data with phase information, such as RF or IQ data. Thorough and ad hoc reanalysis of the acquired data may be performed after the patient has left. The ultrasound imaging system provides the reanalysis without or with export of the ultrasound data. Additionally, a regular PACS system with specially installed software and hardware may also provides the reanalysis with the storing ultrasound data with phase information.

### BACKGROUND

The present embodiments relate to review in ultrasound diagnostic imaging. Data may be reprocessed for the review of previous examinations.

In diagnostic ultrasound systems, a clip of video data is stored for review. Video data is envelope detected, log compressed, and scan converted data output for display. Due to the processing to generate the video data, some of the original information, such as phase information and dynamic range (16˜20 bits data versus 8 bits data), is not saved. There may be no way to change the image processing or formation using the stored video data.

Other ultrasound imaging systems store data that is pre-scan converted and that has not gone through some other post processing. The settings for the skipped post processing, such as edge enhancement, display maps for B mode imaging, and persistence and spatial smooth filters for color flow imaging, may be set differently for the review. However, the data stored is log compressed and does not include phase information.

For more sophisticated processing from radio frequency (RF) or in-phase and quadrature (IQ) data, additional hardware or software collects the appropriate data during the examination. The collected data is exported for processing and review on a personal computer or review station. Real dynamic range change, clutter filter change, signal-to-noise (SNR) change by low pass filters, or other processing, such as strain imaging, may be performed on the RF or IQ data.

### BRIEF SUMMARY

By way of introduction, the preferred embodiments described below include methods, computer readable media and systems for raw data reprocessing and storing data for raw data reprocessing. Clip storing and review functions are more versatile by storing ultrasound data with phase information, such as RF or IQ data. Reanalysis of the acquired data may be performed after the patient has left. The ultrasound imaging system provides the reanalysis without or with export of the ultrasound data. A PACS system may provide the reanalysis of the stored ultrasound data with phase information.

In a first aspect, a method is provided for raw data reprocessing in ultrasound diagnostic imaging. Ultrasound data is acquired with a transducer connected with an ultrasound imaging system. The ultrasound data is radio frequency or in-phase and quadrature data. A first image is generated from the ultrasound data in correspondence with the acquisition. The ultrasound data is stored. A second image is generated by the ultrasound imaging system from the stored ultrasound data. A second image may be generated by a PACS system using the ultrasound data.

In a second aspect, an ultrasound imaging system is provided for raw data reprocessing. A receive image former is operable to generate radio frequency or in-phase and quadrature ultrasound data in response to acoustic echoes. A memory is operable to store the radio frequency or in-phase and quadrature ultrasound data. An image processor is operable to generate a first image from the radio frequency or in-phase and quadrature ultrasound data and is operable to generate later a second image from the radio frequency or in-phase and quadrature ultrasound data.

In a third aspect, a computer readable storage medium has stored therein data representing instructions executable by a programmed processor for raw data reprocessing in ultrasound diagnostic imaging. The storage medium includes instructions for: scanning a region of interest with acoustic energy; storing ultrasound data including samples with phase information; processing the ultrasound data as a function, in part, of the phase information, the processing being part of a review function; and generating a first image as a function of the processing of the ultrasound data.

In a fourth aspect, a method is provided for storing data for raw data reprocessing in ultrasound diagnostic imaging. Coherent ultrasound samples are acquired. An image is generated from the coherent ultrasound samples. A DICOM file with the image in a public element and the coherent ultrasound samples in a private element is stored. The DICOM file may be read by a PACS system which can extract the coherent ultrasound samples from the private element and generate a second image.

In a fifth aspect, a method is provided for raw data reprocessing in ultrasound diagnostic imaging. A multi-dimensional region is scanned with pulsed Doppler firings. Radio frequency or in-phase and quadrature information responsive to the scanning is stored. During review, placement of a pulse Doppler gate anywhere within the multi-dimensional region is allowed.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound imaging system for raw data reprocessing;

FIG. 2 is a block diagram of another embodiment of an ultrasound imaging system for raw data reprocessing;

FIG. 3 is a block diagram of a DICOM encoder for use with the ultrasound imaging system of FIG. 2;

FIG. 4 is a flow chart diagram of one embodiment of a method for raw data reprocessing with an ultrasound imaging system;

FIG. 5 is a flow chart diagram of raw data reprocessing for color flow or Doppler according to one embodiment;

FIG. 6 is a flow chart diagram of a method for a method for raw data reprocessing for B-mode according to one embodiment;

FIG. 7 is a graphical representation of one embodiment of a scan pattern;

FIG. 8 is a flow chart diagram of a method for storing raw data for spectral Doppler imaging according to one embodiment;

FIG. 9 is a flow chart diagram of a method for raw data reprocessing for spectral Doppler imaging according to one embodiment; and

FIG. 10 is graphical representation of one embodiment of a scan pattern.

### DETAILED DESCRIPTION OF THE DRAWINGS AND PRESENTLY PREFERRED EMBODIMENTS

The ultrasound diagnostic imaging system saves IQ or RF data. Herein RF may be beamsummed RF data or channel RF data pre-beam sum. During imaging, cine loop or data review, the saved RF or IQ data may be processed through any appropriate mid-processing and post processing. For example, the saved RF or IQ data may be reprocessed with different mid-processing and/or post processing through the same or different signal paths. During review, different clutter filtering may be selected to focus on a different type of flow. These applications may not be available with video data only clip store. The versatile review is available for further understanding or diagnosis of the collected data. Imaging and data acquisition are separated. Desired analysis not anticipated during acquisition, such as elastography or speckle density analysis, may be performed with the stored data, increasing the productivity and efficiency of ultrasound examination.

In one further embodiment, a Doppler gate is placed anywhere inside a region of interest during review, even if not positioned at the same location during acquisition of the ultrasound data. In another embodiment, the RF or IQ data is stored as a private element in a DICOM file. This enables either a standard review through a traditional workstation or an advanced workflow and analysis through an upgraded or new workstation.

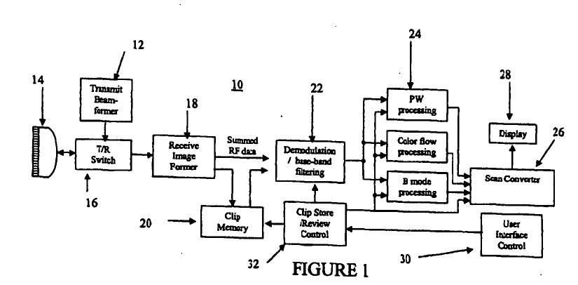

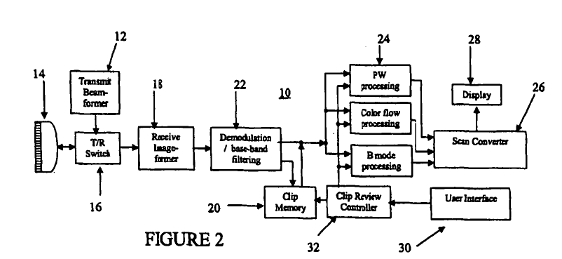

FIGS. 1 and 2 show ultrasound imaging systems 10 for raw data reprocessing. FIG. 1 is a system architecture with RF data saving capability, and FIG. 2 is a system architecture with IQ data saving capability. The ultrasound imaging systems include full size, cart based, handheld or portable imaging systems. The ultrasound imaging systems 10 are used for medical diagnosis.

The systems 10 include a transmit beamformer 12, a transducer 14, a transmit/receive switch 16, a receive image former 18, a memory 20, a demodulator and filter 22, an image processor 24, a scan converter 26, a display 28, a user interface 30 and a review controller 32. Additional, different or fewer components may be provided.

The transmit beamformer 12 generates relatively delayed and apodized electrical waveforms. The waveforms are applied through the transmit/receive switch 16 to the transducer 14 in a plurality of channels of a transmit aperture. The transducer 14 generates a beam or wavefront of acoustic energy in response to the electrical waveforms. The transducer 14 converts acoustic echoes responsive to the transmission to electrical signals for a plurality of elements in a receive aperture. The received electrical signals are provided to the receive image former 18 by the transmit/receive switch 16.

The receive image former 18 is a receive beamformer. Delays, phase rotators, amplifiers and/or other components relatively delay and apodize the electrical signals from the different channels. The delayed and apodized information is summed to form a sample for each spatial location along one or more scan lines. In an alternative embodiment, the receive image former 18 is a processor for fast Fourier transformation. For example, samples for different spatial locations are generated in response to a plane or weakly focused transmit wavefront by processing the received signals in the frequency domain.

The receive image former 18 outputs digital or analog beamformed data. The ultrasound data is either radio frequency or in-phase and quadrature ultrasound data responsive to acoustic echoes. The position of the demodulator and filter 22 determines the type of data output.

The demodulator and filter 22 are mixers and low pass filters. Prior to mixing, the beamformed data is radio frequency data. The beamformed data is mixed to an intermediate or base band frequency by different mixers 90 degrees out of phase. After the mixing, the beamformed data is in-phase and quadrature data. The low pass filters remove or reduce higher frequencies.

The memory 20 is a RAM, hard drive, optical, tape, removable media, flash and/or other now known or later developed memory. The memory 20 is formatted as a clip or CINE memory, but other formats may be used. Ultrasound data for a single scan or multiple scans are stored in the memory 20. Each scan corresponds to one or more sweeps for generating an image. For a B-mode, one or more sweeps scan the region. For color flow mode, each scan line is insonified multiple times in a scan.

In FIG. 1, the memory 20 has an input connected with the receive image former 18 and an output connected with the demodulator and filter 22. Alternatively, the memory 20 receives data from before or within the image former, such as associated with pre-beamsummed RF data. In FIG. 2, the memory 20 has an input connected with the demodulator and filter 22 and an output connected with the image processor 24. Different connections may be provided. The different inputs provide the radio frequency or in-phase and quadrature ultrasound data to the memory for storage.

Beamformed RF or IQ data operable to be processed for B-mode, color flow (CF), and Pulse Doppler (spectral Doppler) is saved into the memory 20. The storage occurs automatically or in response to initiation of storage by the user. The clip memory is large enough to save RF or IQ data over a desired time period, such as over one or more heart cycles or around 6 to 9 seconds. For 3 seconds of data at a 20 Hz frame rate with 8 bits scan converted data and data size of 512×480 for imaging depth 200 mm, the video clip data uses 14M bytes of memory. For IQ data, with a 10 MHz sampling rate, 18 bit accuracy, 20 Hz frame rate, and 250 beams in azimuth, 3 seconds of data uses 223M bytes of memory. For RF data, at least two times more memory compared to IQ data may be used. The RF or IQ data is stored without any compression, but compression may be used. Another possibility is to use 16, 15 or other number of bits of IQ data instead of 18 bit IQ data, saving some extra memory (e.g., using 148 Mbytes). Larger or smaller amounts of memory may be provided, such as using different amounts of time, sample bit sizes, numbers of beams, and/or frame rates.

The memory 20 may additionally store video data. For example, video data output to the display 28 synchronized or corresponding to the stored RF or IQ data is also stored in the memory 20. Alternatively, a different memory stores video data with or without synchronization. In another alternative embodiment, video data is not stored. The settings used to generate any video may or may not be stored.

To recall or use the saved data, the user interface 30 or a control processor sends a request to the review controller 32. The review controller 32 is a general processor, digital signal processor, control processor, application specific integrated circuit, field programmable gate array, digital circuit, analog circuit or combinations thereof. The review controller 32 causes the memory 20 to store data and output stored data, such as outputting the stored data for review imaging of the clip at a desired frame rate. The data is output to the demodulator and filter 22 or the image processor 24.

The output data from the clip memory 20 is processed again or processed for a first time. Different or the same processing settings may be used. The settings are provided by the user interface 30 or automatically. The review controller 32 or a different controller provide the settings to the image processor 24, scan converter 26, receive image former 18 and/or the transmit beamformer 12. The settings include values for adjustable or settable parameters used to generate an image from IQ or RF data by the imaging system.

The image processor 24 is a detector or estimator. For example, the image processor 24 includes a B/M-mode detector, a color flow or Doppler detector, and/or a spectral Doppler detector (PW mode). The detectors are hardware devices, such as dedicated processors or application specific integrated circuits, software devices, such as software running on a general or digital signal processor, or both hardware and software devices. The color flow or Doppler detector includes a clutter filter, estimator and thresholder in one embodiment. The spectral Doppler detector includes a fast Fourier transform processor in one embodiment. Additional, different or fewer detectors may be used.

The image processor 24 may include other image processing components. For example, the image processor 24 includes a three/four-dimensional image data processor, filters (spatial and/or temporal), maps, look-up tables, edge enhancement filters, correlators, extended field of view processors, speckle tracking, strain rate processing, combinations thereof, or other now known or later developed image processing devices for generating an image from IQ or RF data.

The image processor 24 may also include the scan converter 26. Alternatively, the scan converter 26 is a separate component, such as an application specific integrated circuit, processor or field programmable gate array. The scan converter 26 converts data in a scan format to a display format, such as from a Polar coordinate format to a Cartesian coordinate format. The scan converter 26 and/or the image processor 24 may also include a look-up table or processor for mapping intensities or estimates to colors or gray scale levels. Combinations of data from different images or sources may also be performed, such as adding text and graphics to an image.

The image processor 24 with or without the scan converter 26 is operable to generate an image from the radio frequency or in-phase and quadrature ultrasound data. For example, the image processor 24 and scan converter 26 generate a B-mode and/or color flow mode image from the beamformed data. The estimation or detection may remove the phase information associated with the data. One image may be generated contemporaneously with the acquisition of the data by scanning. For example, the image is generated in a same examination, while the patient is present, while the transducer is being used or substantially in real-time with the scanning. The same or different components of the image processor 24 and/or scan converter 26 are operable to generate later another image from the same radio frequency or in-phase and quadrature ultrasound data output by the memory 20.

The images are displayed on the display 28. Alternatively, the images are transmitted to another system or over a network for display on a different device or system.

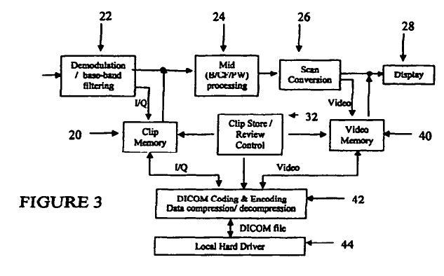

FIG. 3 shows an optional portion of the system of FIG. 2. The system optionally includes a video memory 40, a DICOM encoder 42 connected with the memory 20, and an additional memory 44. Additional, different or fewer components may be provided, such as storing DICOM files in the video memory 40 or the memory 20 without the memory 44.

The video memory 40 stores images output by the scan converter 26. The images are stored in a CINE or other format. The images are represented by video data, such as RGB values.

The DICOM encoder 42 is a processor, application specific integrated circuit, field programmable gate array or other device for encoding or decoding data in a DICOM format. The DICOM encoder 42 may also be operable to compress or decompress data.

The DICOM encoder 42 is operable to encode the RF or IQ ultrasound data in a private element of a DICOM file, and/or video data in a public element of the DICOM file. For example, both RF or IQ data and the corresponding video output data are stored in the memory 44 after encoding. The saved RF or IQ data in the clip memory 20 is combined with corresponding video data by the DICOM encoder 42. The clip memory 20 stores any current collected coherent (IQ or RF) data. For reviewing of previous data sets, the review controller 32 loads data through the DICOM encoder 42 from the memory 44, such as a hard drive memory and driver. The loaded data is stored in the clip memory 20. The memory 20 is large enough for at least two data sets. Alternatively, the data loaded from the DICOM encoder 42 overwrites any data in the memory 20.

The regular video data is stored as standard data elements and corresponding RF or IQ data is stored as private data elements. Data synchronization, encoding and compression are applied to RF or IQ data independently with a privately defined format. This allows the saved data files to be compatible with most standard PACs systems. Software upgraded PACs systems, a specialized PACs system or the imaging system allow utilization of the saved RF or IQ data in the DICOM file. For example, customers may be able to do elastography imaging with the RF or IQ data and review the original or video B mode images side by side with the elastography images for breast application.

The private data element storage of RF or IQ data in the DICOM file may be the standard saving format for both output data storage (PACs system usage) on the memory 44 and/or internal on system clip review data storage on the memory 20. The DICOM encoder 42 processes (encodes and decodes) for the clip store and review procedures. Alternatively, different formats are used for the different memories 20, 40, 44.

For review, the ultrasound imaging system or a medical image archive system (e.g., a PACs system) may generate an ultrasound image from the coherent data of the private element. The image is generated with the same or different processing implemented on a different system or at a different time. For review on a medical image archive system, software or hardware implement different processes, such as detection or filtering.

The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are execute in response to one or more sets of instructions stored in or on computer readable storage media. The functions, acts or tasks are independent of the particular type of instructions set, storage media, processor or processing strategy and may be performed by software, hardware, integrated circuits, filmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

In one embodiment, a computer readable storage medium has stored therein data representing instructions executable by a programmed processor for raw data reprocessing in ultrasound diagnostic imaging. The instructions control scanning of a region of interest with acoustic energy. An image for display is generated from the ultrasound data contemporaneously with the scanning. The image is generated as a function of settings of an ultrasound imaging system, such as dynamic range, filter, threshold, detection or other settings. Alternatively, the scan is performed and data acquired without generating an image. Ultrasound data including samples with phase information are stored. In one embodiment, the samples are stored as a private element of a DICOM file. During later review, the stored ultrasound data is processed as a function, in part, of the phase information. The review images are generated as a function of a setting of a phase related parameter different than originally generated images. Different settings for phase related and/or non-phase related processing may be used. The review image is generated as a function of the processing of the ultrasound data. For example, a clip of images is originally generated and a subsequent clip of images is later generated during review from the same ultrasound data. As another example, some of the data acquired during the scan is not used for generating the contemporaneous images but may be used to allow more versatility during review. A same or even different type of image as the original image is generated for review. The same type of image, but associated with a different spatial location, may be used, such as associated with repositioning a Doppler gate.

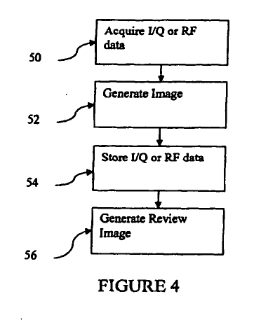

FIG. 4 shows a method for data reprocessing in ultrasound diagnostic imaging. The method includes processes for storing data for reprocessing in ultrasound diagnostic imaging and for reviewing the stored data. The method is implemented with one of the systems described herein or a different system. Additional, different or fewer acts may be provided. For example, the method does not include act 52 or act 56.

In act 50, coherent ultrasound samples are acquired. Coherent ultrasound samples include phase information, such as RF or IQ data. The ultrasound samples are not detected. Detection may remove the phase information, resulting in incoherent data. The coherent ultrasound data is acquired with a transducer connected with an ultrasound imaging system. The data is received in response to transmissions from the transducer.

The coherent ultrasound data is acquired as a set of data representing a plurality of scan lines, such as a single scan in a linear, sector or Vector® format. For B-mode scanning, data for one or more transmissions and receptions along each scan line is obtained. For color flow imaging, data corresponding to multiple transmissions and receptions along each scan line in a region of interest is obtained. The region of interest is a two or three-dimensional region of interest. For spectral Doppler ultrasound data, the data corresponds to multiple transmissions and receptions (pulsed Doppler firings) for a single scan line or one or more range gate locations. Spectral Doppler ultrasound data may have a greater pulse repetition interval than color flow data, such as 20 or more pulses as compared to 2-20 pulses for each scan line. In one embodiment, pulsed Doppler firings are performed for a plurality of scan lines, such as a multi-dimensional region.

In act 52, an ultrasound image is optionally generated from the ultrasound data in correspondence with the acquisition. As the ultrasound data is acquired in act 50, ultrasound images are generated substantially in real-time. The images are generated from the coherent ultrasound samples. The images represent a sequence of all or some images during an examination. For example, the images correspond to one or more heart cycles. A single image may be generated.

The image or images are generated as a function of settings of the ultrasound imaging system. For example, the user configures the system to generate B-mode, color Flow mode and/or spectral Doppler images with settings appropriate for a given application. The settings are provided by application groupings of settings and/or individual adjustments.

In one embodiment, the settings are for generating a color flow image. The desired clutter filter, autocorrelation and threshold functions are selected for two- or three-dimensional velocity, energy, and/or variance imaging. The clutter filter may be set to isolate or emphasize information at different frequency bands. Tissues more likely move at lower frequencies. Different types of flow are associated with different higher frequencies. Different parameters of the autocorrelation or other function to estimate velocity, energy or variance may be set, such as setting a speed of sound in the tissue being imaged. Different thresholds, such as energy thresholding for velocity information, may remove undesired signals. Other settings may be used for color flow image, such as base band filtering settings.

In another embodiment, the settings are for generating a B- or M-mode image. The base band filter isolates or emphasizes information at a desired frequency band. For example, the base band filter isolates information at a fundamental transmit frequency band or a harmonic (e.g., second harmonic) of the fundamental transmit frequency band. The bandwidth may also be set. The detection is responsive to settings. For example, the type of detection, such as intensity or power, may be selected. Other settings may be used for B- or M-mode imaging, such as an amount of synthetic line interpolation (coherent combinations of data) or positioning of an M-mode line in a B-mode image.

The settings determine a sampling rate of the ultrasound data along a range dimension. The sampling rate increases or decreases the resolution. For example, the ultrasound data is acquired with a sampling rate corresponding to dynamic range, frequency and depth settings. Other settings may be used, such as gain.

In another embodiment, the settings are for generating a spectral Doppler image for a gate location along a scan line. Fast Fourier transforms are applied to ultrasound data associated with the gate location. The settings include the location of the gate along the range and azimuth dimensions. Other settings include thresholds for the frequency, power or variance information and display dynamic range.

The image or images generated contemporaneously with the acquisition of the ultrasound data use all or less than all of the acquired ultrasound data. For example, the region is scanned with one density of scan lines. Ultrasound data from a fewer number of scan lines is used to generate the image. As another example, a spectral Doppler image is generated from the ultrasound data associated with one scan line or gate location. Ultrasound data for spectral Doppler analysis may be acquired for other scan lines or gate locations as well.

In act 54, the ultrasound data is stored. The storage of the ultrasound data occurs before, during or after generation of the image in act 52. The stored ultrasound data is responsive to a sequence of scans, such as associated with a clip of data. Alternatively, the stored ultrasound data corresponds to data for a single image. The ultrasound data includes phase information, such as including coherent data (e.g., IQ or RF data).

Other types of ultrasound data may also be stored. For example, video data or incoherent data prior to scan conversion is stored. The data storage is separate, such as in a separate memory or in different locations of a same memory. The different types of data are unsynchronized or are synchronized. For example, video or other data generated from a set of IQ or RF data is synchronized with the IQ or RF data. A time stamp, header, table or other reference indicates the relationship of the types of data. Alternatively, the ultrasound data with phase information and video data are stored together. For example, coherent ultrasound data is stored as a private element of a DICOM file, and video data is stored in a public element of the same DICOM file.

In act 56, an image is generated from the stored ultrasound data. The same ultrasound system used for acquiring the ultrasound data or a different system (e.g., a PACs or another imaging system) generates the image. The image is generated from ultrasound data that includes phase information. The image is a single image or is generated as part of a sequence of images. For example, a previously generated sequence of images is reviewed. For the review, the same or different images are generated from the stored ultrasound data.

The review image or images are generated as a function of settings, such as settings of the ultrasound imaging system. In one embodiment, the settings used to generate the original images are stored. Alternatively, the previous settings are not stored. The same or different settings may be used. New settings allow review that is more versatile. The user or system selects the settings. The different settings result in different processing and/or the use of different ultrasound data. A review image is generated from the same data or from ultrasound data stored but not used to generate an image during the acquisition. For example, a review spectral Doppler image has a gate at a different range on a same scan line or the gate along a different scan line as a previous image.

Different settings may be used to process the ultrasound data differently as a function of the stored phase information. For example, a review image is generated with a different clutter filter, autocorrelation function or clutter filter and autocorrelation functions. To focus more on the slow flow during the clip review rather than faster flow during the data acquisition, the user chooses a lower cutoff of the high pass clutter filter and/or a lower velocity threshold. The user choice is implemented by control panel input or selection of a lower flow application.

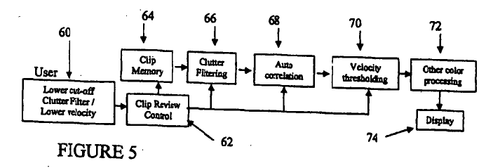

FIG. 5 shows one example for flow or tissue motion imaging review. The user selects the desired clutter filter and/or threshold settings in act 60. The clip review is controlled in act 62 to select the IQ or RF ultrasound data from the memory in act 64. As shown, IQ data is retrieved from the memory. The desired clutter filtering 66, autocorrelation or estimation 68 and thresholding 70 are applied. Other color processing may also be applied in act 72, such as a different persistence control, a different color flow map or color-mapping scheme, or a color post-smoothing scheme for velocity and energy display. While not shown, the other processing may also be controlled in act 72 based on new settings. The image is displayed in act 74.

The review image may be generated with different base band filter, detection or base band filter and detection settings. For example, the original image is generated for fundamental frequencies, but the review image is generated for harmonic frequencies where the stored ultrasound data includes the desired frequency information. In another example, different base band filter settings provide for a different SNR and resolution trade off during B-mode clip review. Another example, the dynamic range, synthetic line interpolation or edge enhancement settings are changed for review.

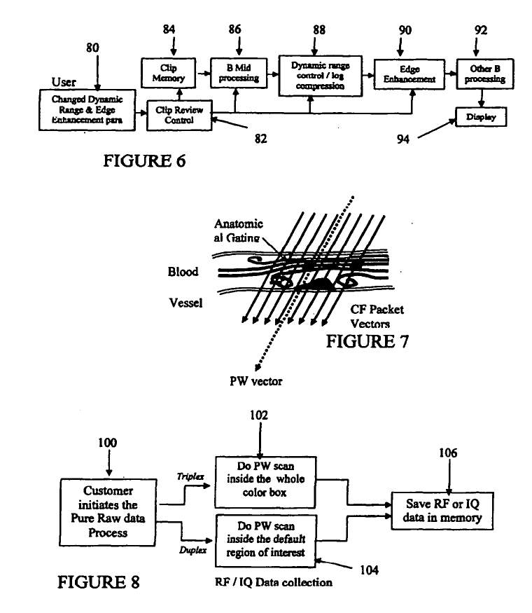

FIG. 6 shows a user or system-based change in the dynamic range and edge enhancement in act 80. For example, the user changes the dynamic range and edge enhancement settings in the review mode as compared to settings used during acquisition. The clip review is controlled in act 82 to select the ultrasound data and configure the processing path based on the settings. The processing path in this example is a B-mode path. In act 84, the stored clip of RF or IQ ultrasound data is loaded into the path. Base band filtering and detecting 86, dynamic range and log compression 88 and edge enhancement 90 are performed. Other B-mode processing, such as application of a selected gray scale map, may be performed in act 92. While not show, the other processing may also be controlled in act 82 based on new settings. In act 94, the review image is displayed.

The review image may be generated as or allow a better zoomed image. Rather than interpolating data in range on detected data, which may not provide any more resolution and just smoothes the image, the ultrasound data is resampled along the range dimension from the IQ or RF data. By increasing the sampling rate, an increased range resolution may be provided for the zoom image. With the saved RF or IQ data, whenever the customer starts the zoom function, the resampling in the zoom region matches the display window size and satisfies the Nyquist sampling rate. If the resampling reaches an upper limit, the data may be interpolated or resolution does not increase further. Along lateral dimensions, the ultrasound data may be coherently interpolated. The phase information is used to generate ultrasound data representing a scan line. Upsampling with coherent interpolation followed with integer or fractional resampling may keep the line density sufficient for the Nyquist requirement in the lateral dimension. As a result, no resolution loss and possible resolution increases are provided during the review.

The review image may be a spectral Doppler image. For example, a spectral Doppler image associated with a same gate location as the original is generated with the same or different settings. Since the spectral or pulse Doppler review image is generated from IQ or RF data, the review image may be associated with a different gate location, such as a different gate location along a same scan line used for the original gate. FIG. 7 shows a plurality of color flow mode scan lines and a single pulsed wave vector. The original spectral Doppler image is generated from data along the pulsed wave vector. The stored ultrasound data includes information from different ranges along the vector. For review, the gate is positioned anywhere along the vector. The different position allows analysis of the velocity, flow profile or other flow characteristic at different positions along the PW beam. Since the RF or IQ data for multiple spatial locations, such as a portion or the entire PW beam is stored, the gate may be repositioned to better account for body movement.

For increased versatility, the gate location is positioned anywhere within a color region of interest or at any location for which color flow mode data is stored. In FIG. 7, the CF vectors represent the color region of interest for which color flow ultrasound data is stored. The spectral Doppler image is generated during review from the color flow data. Multi-gating (i.e., more than one spectral Doppler gate) or anatomical gating (i.e., gate positioned as a function of anatomical structure) is provided during review even if not used during acquisition. For example, gates are positioned in the middle of the vessel before or after a stenosis to track the velocity change per stenosis effect even if not done during acquisition. The color flow data with phase information is treated as PW data with a pulse repetition interval (PRI) equal to integer times (1, 2, . . . , N) of the color flow operating PRI. FFT spectrum analysis is applied to the CF vector data for each gate. B-mode data may alternatively be used for generating spectral Doppler review images. The B-mode data is associated with a lower PRI which is the B-mode frame rate at a maximum.

During review, M-mode images may be generated from the stored RF or IQ data acquired for M-mode, B-mode, spectral Doppler mode and/or color flow mode imaging. The saved RF or IQ data is treated as representing M-mode vectors with a PRF determined by the acquisition PRF or frame rate. During the clip review, the M-mode vector is any of the vectors from other modes or an arbitrary line, such as an anatomical line that spreads in the azimuth direction.

In other embodiments, increased versatility is provided by acquiring data that may be later used to generate review images even if not used to generate an image during acquisition. For example, color flow or M-mode ultrasound information is stored without generating a color flow or M-mode image respectively. As another example, spectral Doppler data is acquired for a plurality of scan lines even if only a portion or none of the data is used for imaging during acquisition. The storage of ultrasound data with phase information for a plurality of locations allows, during review, placement of a pulse Doppler gate anywhere within the scanned region, such as a multidimensional region. Any spectral Doppler image generated contemporaneously with the scanning has a same or different location for the pulse Doppler gate. A spectral Doppler review image is generated as a function of the RF or IQ information.

FIG. 8 shows two different approaches for acquiring ultrasound data to be used for later review but not necessarily during current imaging. For example, extra M-mode information is acquired. As another example, extra spectral or pulsed Doppler information is acquired. FIG. 8 shows an example for acquiring extra spectral or pulsed Doppler information.

In act 100, the user or system initiates storage of the coherent ultrasound data. For example, once the system is acquiring data from a desired location and with desired settings, the user initiates storage of the resulting ultrasound data. The imaging during acquisition may be associated with scanning for a single imaging mode, a duplex imaging mode (i.e., two or more imaging modes, such as B-mode and spectral Doppler mode), or a triplex imaging mode (i.e., three imaging modes, such as B-mode, color flow mode and spectral Doppler).

In act 102, the storage of ultrasound data is initiated as part of a triplex scan. The scan process is altered for the pulse or spectral Doppler mode. The pulse Doppler scan format covers a region instead of one or a limited number of scan lines. For example, the entire region for color flow mode is scanned with a pulse Doppler PRF and a relatively larger FSC (e.g., if the PRF is 1 KHz and 3 seconds data is needed, the FSC equals 3000). The system may scan inside the color box with an acoustic beam grid sparser than the B-mode or color flow acoustic beam grids. FIG. 10 shows an example of the relative grids. Group interleaving allows acquisition of data for the different modes. For each group of pulse Doppler beam firings, RF or IQ data is acquired over a period of time, such as one heart cycle. By increasing the spectral Doppler scan region, the Doppler gate may be placed anywhere inside the region of interest during the review. In one embodiment, the region of interest is a color box, but may be a default region of interest around the pulse Doppler gate.

In act 104, the storage of ultrasound data is initiated as part of a duplex scan. The scan process is altered to have a similar firing sequence and firing scheme as described above in Triplex mode. The pulse Doppler beam scan region is a region of interest around the current PW gate, such as a 2 cm×2.5 cm region for range and azimuth, respectively. A user selected or other region may be used.

In act 106, the RF or IQ data is stored. The stored data includes information not necessarily used for imaging during acquisition. During review, the Doppler gate may be positioned anywhere inside the region for which spectral Doppler information was acquired. The gate placement may be limited to the Doppler acoustic beam grid, such as shown in FIG. 10. Since RF or IQ data is saved for the different locations, the parameters for pulsed Doppler processing may be changed during review. The PRF may only be integer divisions of the collecting or original PRF. Anatomical gating or multi-gating maybe provided at regular pulse Doppler PRF.

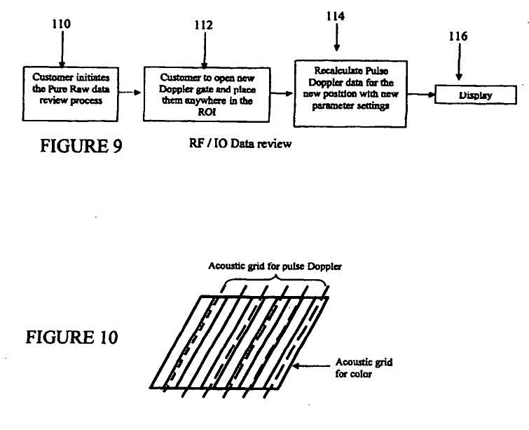

FIG. 9 shows use of the ultrasound data. In act 110, the review is initiated. A new Doppler gate or M-mode line is positioned in act 112. The position is within a region for which spectral Doppler or M-mode data was acquired and stored. In act 114, the ultrasound data is processed to generate an image associated with the new position. The image is displayed in act 116.

The review may assist in diagnosis. Research may benefit from the versatility of the ultrasound imaging system with coherent data storage. Since both magnitude and phase information are saved in RF or IQ data, more sophisticated processing may be performed with the saved clip data. Doctors may review the saved data in different formats than originally displayed, such as reviewing a B-mode image and deriving a previously undisplayed elastography image derived from the RF or IQ data. Acquisition is separated from analysis. There may be a reduction in acquisition time and corresponding reductions in repetitive stress injury since only the settings may need to be changed, not a repeat scan of the patient.

The RF data may be beamformed RF data, or may be pre-beamformed channel RF data. In case of pre-beamformed or channel RF data, the saved RF data may be used for phase aberration or split aperture receive compounding during review or in post analysis.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for raw data reprocessing in ultrasound diagnostic imaging, the method comprising:
acquiring ultrasound data with a transducer connected with an ultrasound imaging system, the ultrasound data being radio frequency or in-phase and quadrature data;
generating a first image from the ultrasound data in correspondence with the acquisition;
storing the ultrasound data; and
generating with the ultrasound imaging system a second image from the stored ultrasound data.

2. The method of claim 1 wherein generating the first image comprises generating the first image as part of a first sequence of images, wherein storing comprises storing the ultrasound data for the first sequence, and wherein generating the second image comprises generating a second sequence from the ultrasound data of the first sequence as a review of the first sequence.

3. The method of claim 1 wherein storing comprises storing the first image synchronized with the ultrasound data, the first image stored as video data.

4. The method of claim 1 wherein storing comprises storing the ultrasound data as a private element of a DICOM file.

5. The method of claim 1 wherein generating the first imaging comprises generating as a function of first settings of the ultrasound imaging system, and wherein generating the second image comprises generating as a function of second settings of the ultrasound imaging system, the second settings different than the first settings.

6. The method of claim 1 wherein acquiring comprises acquiring the ultrasound data as a set of data representing a plurality of scan lines, wherein generating the first image comprises generating the first image as a function of less than all of the ultrasound data of the set, and wherein generating the second image comprises generating the second image from the ultrasound data including data of the set not used for the first image.

7. The method of claim 6 wherein acquiring comprises acquiring spectral Doppler ultrasound data for the plurality of scan lines, wherein generating the first image comprises generating a first spectral Doppler image from the ultrasound data associated with a first one of the scan lines and not associated with a second one of the scan lines, and wherein generating the second image comprises generating a second spectral Doppler image from the ultrasound data associated with the second one of the scan lines.

8. The method of claim 1 wherein generating the first image comprises generating the first image with clutter filter and autocorrelation functions, and wherein generating the second image comprises generating the second image with at least different clutter filter, autocorrelation function or clutter filter and autocorrelation functions.

9. The method of claim 1 wherein generating the first image comprises generating the first image with base band filter and detection settings, and wherein generating the second image comprises generating the second image with at least different base band filter, detection or base band filter and detection settings.

10. The method of claim 1 wherein generating the first image comprises generating the first image with a first sampling of the ultrasound data along a range dimension, and wherein generating the second image comprises generating the second image as a zoomed image with a second sampling of the ultrasound data along the range dimension, the second sampling results in more output samples along the range dimension compared to the first sampling, and with coherent interpolation from the ultrasound data along an azimuth dimension.

11. The method of claim 1 wherein generating the first image comprises generating a first spectral Doppler image for a first gate location along a scan line, and wherein generating the second image comprises generating a second spectral Doppler image from the ultrasound data for a second gate location along the scan line, the second gate location different than the first gate location.

12. The method of claim 1 wherein acquiring comprises acquiring the ultrasound data being color flow data representing at least a two dimensional region of interest, wherein generating the first image comprises generating a color flow image, and wherein generating the second image comprises generating a spectral Doppler image from the color flow data for a range gate location selected during review.

13. The method of claim 1 wherein the ultrasound data is pre-beamsummed channel radio frequency data.

14. An ultrasound imaging system for raw data reprocessing, the ultrasound imaging system comprising:
a receive image former operable to generate radio frequency or in-phase and quadrature ultrasound data in response to acoustic echoes;
a memory operable to store the radio frequency or in-phase and quadrature ultrasound data; and
an image processor operable to generate a first image from the radio frequency or in-phase and quadrature ultrasound data and operable to later generate a second image from the radio frequency or in-phase and quadrature ultrasound data.

15. The ultrasound imaging system of claim 14 wherein the receive image former comprises a receive beamformer, the ultrasound data comprises beamformed data, and the image processor comprises a detector or estimator.

16. The ultrasound imaging system of claim 14 wherein the ultrasound data comprises radio frequency data, and wherein the memory has an output operable to output the stored ultrasound data to a demodulator.

17. The ultrasound imaging system of claim 14 wherein the receive image former comprises a demodulator, and the ultrasound data comprises in-phase and quadrature data.

18. The ultrasound imaging system of claim 14 wherein the memory comprises a clip memory;
further comprising a DICOM encoder connected with the memory, the DICOM encoder operable to encode the ultrasound data in a private element of a DICOM file and data for the first image in a public element of the DICOM file.

19. In a computer readable storage medium having stored therein data representing instructions executable by a programmed processor for raw data reprocessing in ultrasound diagnostic imaging, the storage medium comprising instructions for:
scanning a region of interest with acoustic energy;
storing ultrasound data including samples with phase information;
processing the ultrasound data as a function, in part, of the phase information, the processing being part of a review function; and
generating a first image as a function of the processing of the ultrasound data.

20. The instructions of claim 19 further comprising generating a second image from the ultrasound data contemporaneously with the scanning, the second image part of a clip of images and the first image part of a review clip of images.

21. The instructions of claim 20 wherein generating the second image comprises generating as a function of second settings of a ultrasound imaging system, and wherein generating the first image comprises generating as a function of first settings of the ultrasound imaging system, the second settings different than the first settings.

22. The instructions of claim 20 wherein scanning comprises scanning for possible imaging, wherein generating the first image comprises generating the first image from ultrasound data not used for the second image.

23. The instructions of claim 20 wherein generating the first image comprises generating the first image as a function of a setting of a phase related parameter different than the second image.

24. The instructions of claim 20 wherein generating the first image comprises generating the first image as a same type of image as the second image and the first image associated with a different location within a same region as the second image.

25. The instructions of claim 19 wherein storing comprises storing the ultrasound data as a private element of a DICOM file.

26. A method for storing data for raw data reprocessing in ultrasound diagnostic imaging, the method comprising:
acquiring coherent ultrasound samples;
generating an image from the coherent ultrasound samples; and
storing a DICOM file with the image in a public element and the coherent ultrasound samples in a private element.

27. The method of claim 26 further comprising:
generating with a medical image archive system an ultrasound image as a function of the coherent ultrasound samples from the private element.

28. A method for raw data reprocessing in ultrasound diagnostic imaging, the method comprising:
scanning a multi-dimensional region with pulsed Doppler firings;
storing radio frequency or in-phase and quadrature information responsive to the scanning; and
allowing, during review, placement of a pulse Doppler gate anywhere within the multi-dimensional region.

29. The method of claim 28 further comprising:
generating a spectral Doppler image as a function of the radio frequency or in-phase and quadrature information corresponding to the pulse Doppler gate.

30. The method of claim 29 wherein any spectral Doppler image generated contemporaneously with the scanning has a different location for the pulse Doppler gate.
